(19) United States
(12) Patent Application Publication
(10) Pub. No.: US 2013/0079825 A1
(43) Pub. Date: Mar. 28, 2013

(54) TEMPLATE FOR MULTIPLE LENGTH SPINAL RODS

(71) Applicants: Robert Loke, Memphis, TN (US); Julien Prevost, Memphis, TN (US)

(72) Inventors: Robert Loke, Memphis, TN (US); Julien Prevost, Memphis, TN (US)

(73) Assignee: Warsaw Orthopedic, Inc., Warsaw, IN (US)

(21) Appl. No.: 13/245,505

(22) Filed: Sep. 26, 2011

(51) Int. Cl. A61B 17/70; A61B 17/88

(52) U.S. Cl. 606/259; 606/264; 606/279

(57) ABSTRACT

A spinal rod template has a rod and a plurality of size identifiers. The rod has a first end separated by a length from a second end. The first end may be shaped generally like a spinal rod and configured to be received within a first bone anchor assembly. The size identifiers are oriented along the length of the rod at the second end of the rod. Each of the plurality of size identifiers identifies a specific length for a spinal rod implant. The second end of the rod is configured to be received in a second bone anchor assembly such that the plurality of size identifiers specify specific length for a spinal rod implant based upon the relationship of the second bone anchor assembly to the plurality of size identifiers.

### FIELD OF INVENTION

Embodiments of the invention relate to instrumentation for spinal fixation systems. More particularly, the embodiments relate to templates for spinal fixation systems.

### BACKGROUND

A spinal fixation device generally consists of stabilizing elements, such as rods, attached by anchors to the vertebrae in the section of the vertebral column that is to be stabilized. The anchors are spaced apart into bone and then connected by a span of a rod. It is necessary to properly identify the length of the rod to span the anchors. Determination of the appropriate length of the rod is often accomplished by inserting trial instruments each having an end or a foot of different length to gauge the distance between the bolts or connectors. This trial and error process consumes operating time. Particularly if the rod will span more than two adjacent vertebra, the number of trial instruments required may be cumbersome.

Other instruments may include a device for determining the appropriate length of a rod to be attached to a first bone anchor assembly and a second bone anchor assembly. The device includes a first member having a first end that is capable of being immobilized while the first end is in contact with the first bone anchor assembly. The device also includes a second member having a second end, the second member pivotally coupled to the first member and designed to pivot relative to the first member about a pivot point while the first member is immobilized such that the second end contacts the second bone anchor assembly. The device further includes an indicator capable of indicating the distance between the first end and the second end from which the appropriate length of a rod to be attached to the bone anchor assemblies is determined. These instruments may have some advantages over individual rod templates that are used in a trial and error system, but they may require additional training and may also consume operating time.

The description herein of problems and disadvantages of known apparatuses, methods, and devices is not intended to limit the invention to the exclusion of these known entities. Indeed, embodiments of the invention may include, as a part of the embodiment, portions or all of one or more of the known apparatus, methods, and devices without suffering from the disadvantages and problems noted herein.

### SUMMARY OF THE INVENTION

An embodiment of the present invention provides a spinal rod template having a rod and a plurality of size identifiers. The rod has a first end separated by a length from a second end. The first end may be shaped generally like a spinal rod and configured to be received within a first bone anchor assembly. The size identifiers are oriented along the length of the rod at the second end of the rod. Each of the plurality of size identifiers identifies a specific length for a spinal rod implant. The second end of the rod is configured to be received in a second bone anchor assembly such that the plurality of size identifiers specify specific length for a spinal rod implant based upon the relationship of the second bone anchor assembly to the plurality of size identifiers.

In another embodiment of the invention, a kit of spinal rod templates comprises a first rod having a plurality of size identifiers and a second rod having a plurality of size identifiers. The size identifiers are oriented along the length of the rod. Each of the plurality of size identifiers identifies a specific length for a spinal rod implant. Each of the size identifiers specifies a specific length for a spinal rod implant. At least one of the size identifiers of the first rod is also one of the size identifiers of the second rod.

In yet another embodiment of the invention, a method of sizing a spinal rod is disclosed. The method includes a step of implanting a first bone anchor assembly in a first vertebra and a second bone anchor assembly in a second vertebra. Another step inserts a first end of a rod template into the first bone anchor assembly. A second end of the rod template is inserted into the second bone anchor assembly. The second end of the rod template has a plurality of size identifiers. Another step determines a proper size for a spinal rod implant from a relationship between size identifiers and the second bone anchor assembly.

Additional aspects and features of the present disclosure will be apparent from the detailed description and claims as set forth below.

### BRIEF DESCRIPTION OF THE DRAWINGS

FIG. 1 illustrates a spinal rod implant placed between two bone anchor assemblies affixed to two vertebrae.

FIG. 2 illustrates a side view of an embodiment of a rod template according to an aspect of the invention.

FIG. 3 illustrates a cross section of the rod template of FIG. 2.

FIG. 4 illustrates a top view of the rod template of FIG. 2.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

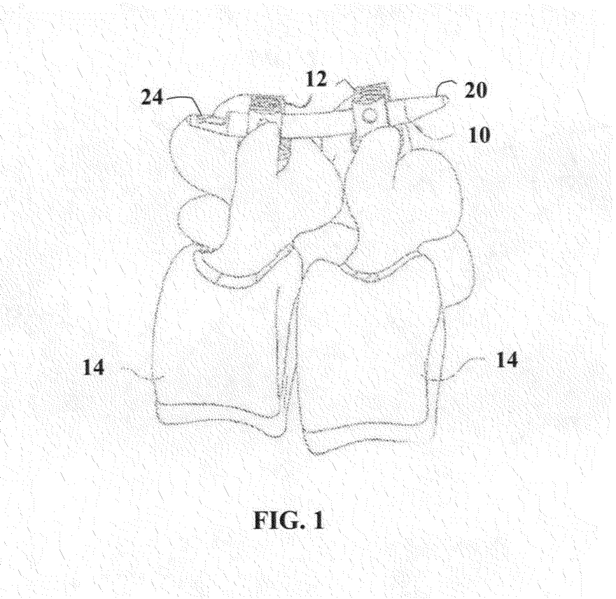

FIG. 1 illustrates a spinal rod implant 10 placed between two bone anchor assemblies 12 affixed to two vertebrae 14. As illustrated, the vertebrae 14 are adjacent, but some applications require a spinal rod implant 10 that extends between bone anchor assemblies 12 affixed to non-adjacent vertebrae 14. Conventionally, a trial and error process is used to determine the distance between two bone anchor assemblies 12. The present invention includes embodiments that may minimize the trial and error necessary to determine the length of a spinal rod implant 110 to be placed between two bone anchor assemblies 12 that are affixed to adjacent or non-adjacent vertebrae 14.

The bone anchor assemblies 12 are conventional and typically include bolts or screws secured to the vertebrae 14, and a connector that securely engages the spinal rod implant 10 to the bolts or screws. The length of the rod is determined by the distance between the bone anchor assemblies 12. Pre-operative images may give a surgeon an estimate of the length of the rod, as well as an estimate of the location of the bone anchor assemblies 12, but the actual location of the bone anchor assemblies 12 and the spinal rod implant 10 may not be final until the surgeon has actually implanted the bone anchor assemblies 12 and performed the other steps of the surgery, all of which may change the actual length of the spinal rod implant 10.

Typically, spinal rod implants 10 are provided in fixed lengths. The indication of the distance between the bone anchor assemblies 12 will enable the user to select to appropriate rod length from the available fixed lengths rods based on criteria desired by the user. A rod template according to an aspect of the present invention may provide an indication of the length of the rod to be placed between bone anchor assemblies 12 in contact with both first end 20 and second end 24 that would include adjustments, such as for extra length needed for affixation to the bone anchor assemblies 12.

As previously mentioned, the bone anchor assemblies 12 need not be on adjacent vertebra. Moreover, the rod and bone anchor assembly system is not limited to two bone anchor assemblies. Additional bone anchor assemblies may be placed between the first and second assemblies. The rod, then, may be connected to the bone anchor assemblies between the first and second assemblies 12.

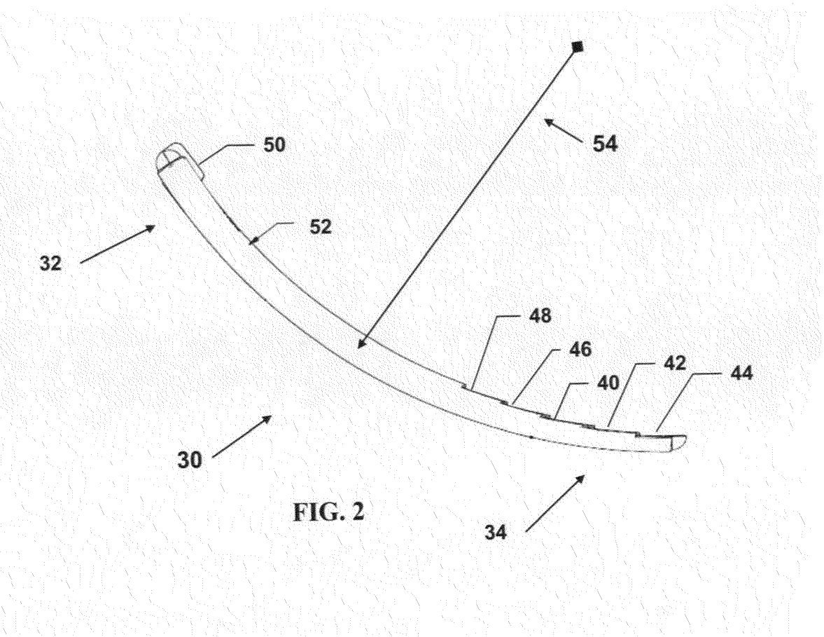

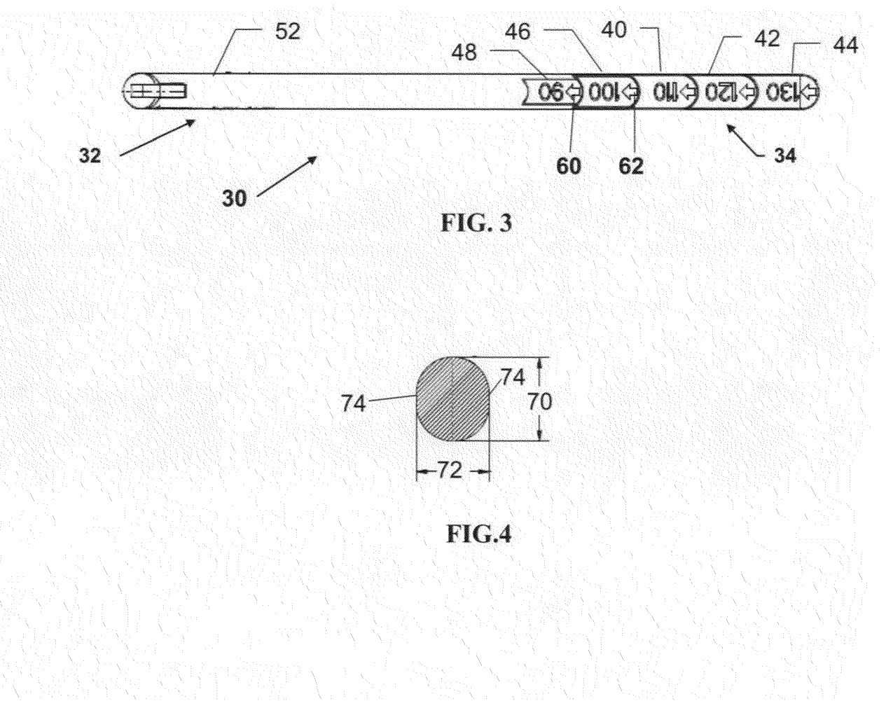

FIG. 2 illustrates a side view of an embodiment of a rod template 30 according to an aspect of the invention. The rod comprises a first end 32 and a second end 34. Size identifiers 40, 42, 44, 46, and 48 (shown more clearly in FIG. 3) are oriented on the second end 34 of the rod template 30. In a preferred embodiment, the central identifier 40 corresponds to the most common length spinal rod implant used in a spinal surgery. The longer identifiers 42 and 44 correlate to the next two sized rods longer than the most common rod. The shorter identifiers 46 and 48 correlate to the next two sized rods shorter than the most common rod.

The identifiers 40-48 are offset in height from one another. The height of the rod template 30 at the first end 32 extends generally uniformly along the length of the rod template 30. Along the length, as each size identifier 40-48 is reached, the height of the rod template 30 diminishes. The shorter identifiers 48 and 46 have a height closer to the height of the first end 32 of the rod template 30, with the shortest identifier 48 having a height closest to the height of the rod template 32. The longer identifiers 42 and 44 have the shortest heights of the rod template 30, with the longest identifier 44 having the shortest height of all the identifiers. The height of the size identifiers 40-48 allow a surgeon to tactilely feel the end of the rod template 30 and determine the length of the spinal rod implant 10 by that feel. Additionally, the change in height, when viewed from the side, allows the surgeon to count the number of identifiers 40-48 from the second end 34 to properly size the length of the spinal rod implant 10. The height of the rod template 30 at the longest identifier 44 may be approximately half the height of the rod template 30 at the first end 32.

In a preferred embodiment, a stop 50 located at the first end 32 of the rod template 30 extends from the first end 32. The stop 50 extends into the bone anchor assembly 12 at the first end 32. The stop 50 makes it difficult to fix the rod template 30 to the bone anchor assembly 12. The stop 50, then, is an added safety feature lessening the likelihood of a surgeon to errantly implant a rod template 30. In addition, laser marks 52 make clearly state “DO NOT IMPLANT” on the rod template 30, or include words such as “TRIAL” to make the surgeon more aware that the rod template 30 is not for implant.

The rod template 30 may extend along a curved path with a fixed radius 54. The curved path of the rod template 30 approximates the curve of the spinal rod implant 10. The path of the rod template 30 may be along a single curve (as shown here with the single radius 54), straight, or along a multi-radius path. The shape of the spinal rod implant 10 dictates the shape of the rod template 30.

Turning now to FIG. 3, FIG. 3 illustrates a top view of the rod template 30 of FIG. 2. The size identifiers 40-48 are shown from a top view. The size identifiers 40-48 may have laser marked numbers to identify the length of the spinal rod implant 10. In this embodiment, the numbers 90, 100, 110, 120, and 130 reference lengths of 90 mm to 130 mm, respectively. Other size numbering systems (such as ordinal numbering systems) may similarly be used as long as the reference on the rod template 30 may be referenced back to a spinal rod implant 10. In another embodiment, color coded bands may be used to determine the length of the spinal rod implant 10. Any system which can delineate along the length of the rod template 30 may be used to implement the rod template and templating method discussed herein. Arrows 60 and 62 may be placed on the identifiers 40-48 to connote the direction of the length readings. For example, if the sizing measurement from the surgeon came between the arrows 60 and 62, then the proper length of the spinal rod implant 10 would be 100 mm.

The identifiers 40-48, arrows 60 and 62, and marks 52 may be laser etched onto the rod template 30. However, the identifiers 40-48, arrows 60 and 62, and marks 52 may instead be painted or created by some other surface process. The identifiers 40-48, arrows 60 and 62, and marks 52 should be visible relative to the rod template 30, but otherwise may be included on the rod template 30 in whatever manner desired.

FIG. 4 illustrates a cross section of the rod template 30 of FIG. 2. The cross section is taken near the first end 32 of the rod template 30. The cross section has a height 70 and a width 72. Generally, the height 70 of the rod template 30 is greater than the width 72 created by flats 74 along the sides of the rod template 30. Again, this approximates the shape of the spinal rod implant 10. The greater height 70 also allows the identifiers 40-48 to be more easily viewable, as the central identifier 40 and the longer identifiers 42 and 44 may extend across almost the entire width of the rod. The width of the identifiers 40-48 is based upon the height of the identifier (because the width is a function of the height). Thus, in FIG. 3, the shortest identifier 48 is less wide than the other identifiers because it has a greater height than the other identifiers and does not extend the full width of the rod template 30.

Rod templates may be included in a kit of rod templates. The kit may include a first rod template having a plurality of size identifiers oriented along the length of the rod. Each of size identifiers may specify a specific length for a spinal rod implant. A second rod may also have a plurality of size identifiers oriented along the length of the rod. Each of the size identifiers identifies a specific length for a spinal rod implant. Each of the size identifiers specifies specific length for a spinal rod implant. The size identifiers on the rods may overlap so that the surgeon may not have to use multiple rod templates in order to find the correct length.

The rod templates of the kit may extend along a curved path to approximate the curve of the spinal rod implant. The rod templates of the kit may have a height extending along the length of the rod to the size identifiers. The height of each of the plurality of size identifiers may be less than the height of the rod. The rods of the kit may include a stop extending from a first end of the rod. The stop may be configured to extend into the bone anchor assembly and prohibit a set screw from engaging the bone anchor assembly.

In use, the surgeon may use the rod template by implanting a first bone anchor assembly in a first vertebra and a second bone anchor assembly in a second vertebra. The first end of the rod template may then be inserted into the first bone anchor assembly. The second end of the rod template may be inserted into the second bone anchor assembly. The surgeon may determine a proper size for a spinal rod implant from a relationship between the size identifiers on the second end of the rod template and the second bone anchor assembly. The surgeon may want the spinal rod implant to end almost immediately after exiting the second bone assembly, or may wish to extend the spinal rod implant farther out the end of the second bone anchor assembly. Because the rod template generally looks like the spinal rod implant, the surgeon may see how the spinal rod implant will generally fit on the bone anchor assemblies by looking at the rod template in the bone anchor assemblies.

The surgeon may use the height and visual cues of the rod template to determine the proper height. The height may provide tactile feedback should the line of sight to the rod template be occluded. Visually, the markings of the size identifiers may inform the surgeon as to the proper length of the spinal rod implant. The height may also be viewed laterally to inform the surgeon of the proper length.

Furthermore, as used herein, the terms components and modules may be interchanged. It is understood that all spatial references, such as “first,” “second,” “exterior,” “interior,” “superior,” “inferior,” “anterior,” “posterior,” “central,” “annular,” “outer,” and “inner,” are for illustrative purposes only and can be varied within the scope of the disclosure.

1. A spinal rod template, comprising:
a rod having a first end separated by a length from a second end, the first end being generally shaped like a spinal rod and configured to be received within a first bone anchor assembly; and
a plurality of size identifiers oriented along the length of the rod at the second end of the rod, each of the plurality of size identifiers identifying a specific length for a spinal rod implant, the second end of the rod being configured to be received in a second bone anchor assembly such that the plurality of size identifiers specify specific length for a spinal rod implant based upon the relationship of the second bone anchor assembly to the plurality of size identifiers.

2. The spinal rod template of claim 1, wherein the length of the rod extends along a curved path.

3. The spinal rod template of claim 2, wherein the curved path approximates the curve of the spinal rod implant.

4. The spinal rod template of claim 1, wherein the plurality of size identifiers includes a central identifier, at least one shorter identifier, and at least one longer identifier.

5. The spinal rod template of claim 4, wherein the at least one longer identifier is closer to the second end than the central identifier.

6. The spinal rod template of claim 5, wherein the rod has a height, the height at the first end extending along the length of the rod to the plurality of size identifiers and wherein the height of the rod at the at least one shorter identifier is greater than the height of the rod at the central identifier.

7. The spinal rod template of claim 1, wherein the rod has a height, the height at the first end extending along the length of the rod to the plurality of size identifiers and wherein the height of each of the plurality of size identifiers is less than the height of the rod.

8. The spinal rod template of claim 1, further comprising a stop extending from the first end of the rod, the stop being configured to extend into the bone anchor assembly and prohibit a set screw from engaging the bone anchor assembly.

9. A kit of spinal rod templates, comprising:
a first rod having a plurality of size identifiers oriented along the length of the rod, each of the plurality of size identifiers identifying a specific length for a spinal rod implant; and
a second rod having a plurality of size identifiers oriented along the length of the rod, each of the plurality of size identifiers identifying a specific length for a spinal rod implant, each of the plurality of size identifiers specifies a specific length for a spinal rod implant;
wherein at least one of the plurality of size identifiers of the first rod is also one of the plurality of size identifiers of the second rod.

10. The kit of claim 9, wherein the length of the rod extends along a curved path.

11. The kit of claim 10, wherein the curved path approximates the curve of the spinal rod implant.

12. The kit of claim 9, wherein the first rod has a height, the height at a first end extending along the length of the first rod to the plurality of size identifiers and wherein the height of each of the plurality of size identifiers is less than the height of the first rod.

13. The kit of claim 9, wherein the first rod further comprising a stop extending from a first end of the first rod, the stop being configured to extend into the bone anchor assembly and prohibit a set screw from engaging the bone anchor assembly.

14. A method of sizing a spinal rod, comprising the steps of:
implanting a first bone anchor assembly in a first vertebra and a second bone anchor assembly in a second vertebra;
inserting a first end of a rod template into the first bone anchor assembly;
inserting a second end of the rod template into the second bone anchor assembly, the second end of the rod template having a plurality of size identifiers; and
determining a proper size for a spinal rod implant from a relationship between size identifiers and the second bone anchor assembly.

15. The method of claim 14, wherein the rod template extends along a curved path.

16. The method of claim 15, wherein the curved path approximates the curve of the spinal rod implant.

17. The method of claim 14, wherein the determining a proper size step comprises reading a visual cue from the size identifiers.

18. The method of claim 14, wherein the rod template has a height, the height at the first end extending along the length of the rod to the plurality of size identifiers, the height of each of the plurality of size identifiers is less than the height of the rod and the height of each of the plurality of size identifiers is different than each other of the plurality of size identifiers, wherein the determining a proper size step comprises receiving tactile feedback form the size identifiers.

19. The method of claim 14, further comprising the steps of:
removing the rod template from the first and second bone anchor assemblies;
choosing a spinal implant rod based upon the proper size; and
implanting the spinal implant rod into the first and second bone anchor assemblies.

20. The method of claim 14, further comprising the step of inserting the rod template in a third bone anchor assembly, the third bone anchor assembly being located in a vertebra between the first bone anchor assembly and the second bone anchor assembly.
